SEQUENCE LISTING (24 sequences)

SEQ ID NO: 1 - DNA, 1297 bp, Homo sapiens
  CDS (69)...(887)
aagcttggcacgaggtggcacgaggcctcgtgccaagcttggcacgaggccgcctggaggcacaggccatgaggggctctcaggaggtgctgctgatgtggcttctggtgttggcagtgggcggcacagagcacgcctaccggcccggccgtagggtgtgtgctgtccgggctcacggggatcctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcgtcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcaggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaa

SEQ ID NO: 2 - protein, 273 aa, Homo sapiens
MRGSQEVLLMWLLVLAVGGTEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 3 - protein, 254 aa, Homo sapiens
TEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 4 - DNA, 284 bp, Homo sapiens
ggcggcggcgcgtgcgcgccccggatccggcggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctatagg

SEQ ID NO: 5 - DNA, 40 bp, Homo sapiens
tgcggcggtaggcggtcctatagatggttcggtaggtgct

SEQ ID NO: 6 - DNA, 18 bp, Homo sapiens
gctgatgtggcttctggt

SEQ ID NO: 7 - DNA, 18 bp, Homo sapiens
ggtaggcgtgctctgtgc

SEQ ID NO: 8 - protein, 708 aa, Homo sapiens
THRGLHISALATYRARGPRGLYARGARGVALCYSALAVALARGALAHISGLYASPPRVALSERGLSERPHEVALGLNARGVALTYRGLNPRPHELETHRTHRCYSASPGLYHISARGALACYSSERTHRTYRARGTHRILETYRARGTHRALATYRARGARGSERPRGLYLEALAPRALAARGPRARGTYRALACYSCYSPRGLYTRPLYSARGTHRSERGLYLEPRGLYALACYSGLYALAALAILECYSGLNPRPRCYSARGASNGLYGLYSERCYSVALGLNPRGLYARGCYSARGCYSPRALAGLYTRPARGGLYASPTHRCYSGLNSERASPVALASPGLCYSSERALAARGARGGLYGLYCYSPRGLNARGCYSVALASNTHRALAGLYSERTYRTRPCYSGLNCYSTRPGLGLYHISSERLESERALAASPGLYTHRLECYSVALPRLYSGLYGLYPRPRARGVALALAPRASNPRTHRGLYVALASPSERALAMETLYSGLGLVALGLNARGLEGLNSERARGVALASPLELEGLGLLYSLEGLNLEVALLEALAPRLEHISSERLEALASERGLNALALEGLHISGLYLEPRASPPRGLYSERLELEVALHISSERPHEGLNGLNLEGLYARGILEASPSERLESERGLGLNILESERPHELEGLGLGLNLEGLYSERCYSSERCYSLYSLYSASPSER

SEQ ID NO: 9 - protein, 31 aa, Homo sapiens
AICQPPCRNGGSCVQPGRCRCPAGWRGDTCQ

SEQ ID NO: 10 - protein, 42 aa, Homo sapiens
SDVDECSARRGGCPQRCVNTAGSYWCQCWEGHSLSADGTLCV

SEQ ID NO: 11 - protein, 256 aa, Homo sapiens
PRLYSGLYGLYPRPRARGVALALAPRASNPRTHRGLYVALASPSERALAMETLYSGLGLVALGLNARGLEGLNSERARGVALASPLELEGLGLLYSLEGLNLEVALLEALAPRLEHISSERLEALASERGLNALALEGLHISGLYLEPRASPPRGLYSERLELEVALHISSERPHEGLNGLNLEGLYARGILEASPSERLESERGLGLNILESERPHELEGLGLGLNLEGLYSERCYSSERCYSLYSLYSASPSER

SEQ ID NO: 12 - protein, 331 aa, Homo sapiens
THRGLHISALATYRARGPRGLYARGARGVALCYSALAVALARGALAHISGLYASPPRVALSERGLSERPHEVALGLNARGVALTYRGLNPRPHELETHRTHRCYSASPGLYHISARGALACYSSERTHRTYRARGTHRILETYRARGTHRALATYRARGARGSERPRGLYLEALAPRALAARGPRARGTYRALACYSCYSPRGLYTRPLYSARGTHRSERGLYLEPRGLYALACYSGLYALAALAILECYSGLNPRPRCYSARGASNGLYGLYSERCYSVALGLNPRGLYARGCYSARGCYSPRALAGLYTRPARGGLYASPTHRCYSGLN

SEQ ID NO: 13 - protein, 158 aa, Homo sapiens
TEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCV

SEQ ID NO: 14 - protein, 73 aa, Homo sapiens
AICQPPCRNGGSCVQPGRCRCPAGWRGDTCQSDVDECSARRGGCPQRCVNTAGSYWCQCWEGHSLSADGTLCV

SEQ ID NO: 15 - protein, 169 aa, Homo sapiens
AICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 16 - protein, 181 aa, Homo sapiens
TEHAYRPGRRVCAVRAHGDPVSESFVQRVYQPFLTTCDGHRACSTYRTIYRTAYRRSPGLAPARPRYACCPGWKRTSGLPGACGAPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 17 - protein, 293 aa, Homo sapiens
MGSRAELCTLLGGFSFLLLLIPGEGAKGGSLRESQGVCSKQTLVVPLHYNESYSQPVYKPYLTLCAGRRICSTYRTMYRVMWREVRREVQQTHAVCCQGWKKRHPGALTCEAICAKPCLNGGVCVRPDQCECAPGWGGKHCHVDVDECRTSITLCSHHCFNTAGSFTCGCPHDLVLGVDGRTCMEGSPEPPTSASILSVAVREAEKDERALKQEIHELRGRLERLEQWAGQAGAWVRAVLPVPPEELQPEQVAELWGRGDRIESLSDQVLLLEERLGACSCEDNSLGLGVNHR

SEQ ID NO: 18 - DNA, 1339 bp, Mus musculus
  CDS (261)...(1094)
gtagggctctgccgggacctgggtcttccctctcctggagctgcagaggccagaagttcagtggtgaggggtccaaggagagtccggggagaccagggaggctctgtccatcccctgtccctgtccctgtgggaagcccccggcagcagcaagacgctggctgttccacctgcccacaagaacagccaccaccagtacccaggggatgacaagcggccggaccacaggccacaaaaagaagaaggctaccccacttacagatgcagaccatgtggggctccggagaactgcttgtagcatggtttctagtgttggcagcagatggtactactgagcatgtctacagacccagccgtagagtgtgtactgtggggatttccggaggttccatctcggagacctttgtgcagcgtgtataccagccttacctcaccacttgcgacggacacagagcctgcagcacctaccgaaccatctaccggactgcctatcgccgtagccctggggtgactcccgcaaggcctcgctatgcttgctgccctggttggaagaggaccagtgggctccctggggcttgtggagcagcaatatgccagcctccatgtgggaatggagggagttgcatccgcccaggacactgccgctgccctgtgggatggcagggagatacttgccagacagatgttgatgaatgcagtacaggagaggccagttgtccccagcgctgtgtcaatactgtgggaagttactggtgccagggatgggagggacaaagcccatctgcagatgggacgcgctgcctgtctaaggaggggccctccccggtggccccaaaccccacagcaggagtggacagcatggcgagagaggaggtgtacaggctgcaggctcgggttgatgtgctagaacagaaactgcagttggtgctggccccactgcacagcctggcctctcggtccacagagcatgggctacaagatcctggcagcctgctggcccattccttccagcagctggaccgaattgattcactgagtgagcaggtgtccttcttggaggaacatctggggtcctgctcctgcaaaaaagatctgtgataacctctcaccacccaggctggatagagcagtcatccctagatcccttgtagccagagttcaggcgctgtctggtggtgcctatgagcagaaggccctgcctcattgtccctctttcttaggaggttcctaggacttgggcatggggagtggggtcttgtgtgactcttcagtggggctccctgtctaagtggtaaggtggggattgtctccatctttgtcataataaagctgagacttga

SEQ ID NO: 19 - protein, 278 aa, Mus musculus
MQTMWGSGELLVAWFLVLAADGTTEHVYRPSRRVCTVGISGGSISETFVQRVYQPYLTTCDGHRACSTYRTIYRTAYRRSPGVTPARPRYACCPGWKRTSGLPGACGAAICQPPCGNGGSCIRPGHCRCPVGWQGDTCQTDVDECSTGEASCPQRCVNTVGSYWCQGWEGQSPSADGTRCLSKEGPSPVAPNPTAGVDSMAREEVYRLQARVDVLEQKLQLVLAPLHSLASRSTEHGLQDPGSLLAHSFQQLDRIDSLSEQVSFLEEHLGSCSCKKDL

SEQ ID NO: 20 - protein, 29 aa, Mus musculus
TCDGHRACSTYRTIYRTAYRRSPGLAPAR

SEQ ID NO: 21 - protein, 32 aa, Mus musculus
QPGRCRCPAGWRGDTCQSDVDECSARRGGCPQ

SEQ ID NO: 22 - protein, 37 aa, Mus musculus
CVPKGGPPRVAPNPTGVDSAMKEEVQRLQSRVDLLEE

SEQ ID NO: 23 - protein, 29 aa, Mus musculus
QQLGRIDSLSEQISFLEEQLGSCSCKKDS

SEQ ID NO: 24 - protein, 255 aa, Homo sapiens
TEHVYRPSRRVCTVGISGGSISETFVQRVYQPYLTTCDGHRACSTYRTIYRTAYRRSPGVTPARPRYACCPGWKRTSGLPGACGAAICQPPCGNGGSCIRPGHCRCPVGWQGDTCQTDVDECSTGEASCPQRCVNTVGSYWCQGWEGQSPSADGTRCLSKEGPSPVAPNPTAGVDSMAREEVYRLQARVDVLEQKLQLVLAPLHSLASRSTEHGLQDPGSLLAHSFQQLDRIDSLSEQVSFLEEHLGSCSCKKDL

(19) United States
(12) Patent Application Publication
(10) Pub. No.: US 2003/0166907 A1
(43) Pub. Date: Sep. 4, 2003

(54) Mammalian neuro-growth factor like protein

(75) Inventors: Paul O. Sheppard, Granite Falls, WA (US); Laura J. Jelinek, Seattle, WA (US)

Correspondence Address: Paul G. Lunn, Esq., ZymoGenetics, Inc., 1201 Eastlake Avenue East, Seattle, WA, 98102, US

(73) Assignee: ZymoGenetics, Inc.

(21) Appl. No.: 09/852,472

(22) Filed: May 10, 2001

Related U.S. Application Data
(63) Continuation of application No. 09/099,295, filed on Jun. 18, 1998, now abandoned.
(60) Provisional application No. 60/050,143, filed on Jun. 18, 1997.

(51) Int. Cl. C07H 21/04; C12P 21/02; C12N 5/06

(52) U.S. Cl. 536/23.5; 435/320.1; 435/325; 435/69.1

(57) ABSTRACT

Novel mammalian Zneu1 polypeptides, polynucleotides encoding the polypeptides, and related compositions and methods including antibodies and anti-idiotypic antibodies.

This is a continuation under 35 U.S.C. §120 of U.S. patent application Ser. No. 09/099,295 filed Jun. 18, 1998, which claims the benefit under 35 U.S.C. §119(e) of U.S. Provisional Application Serial No. 60/050,143 filed Jun. 18, 1997.

### BACKGROUND OF THE INVENTION

Proliferation and differentiation of cells of multicellular organisms are controlled by hormones and polypeptide growth factors. These diffusable molecules allow cells to communicate with each other and act in concert to form cells and organs, and to repair and regenerate damaged tissue. Examples of hormones and growth factors include the steroid hormones (e.g. estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO) and calcitonin.

Hormones and growth factors influence cellular metabolism by binding to proteins. Proteins may be integral membrane proteins that are linked to signaling pathways within the cell, such as second messenger systems. Other classes of proteins are soluble molecules, such as the transcription factors.

### SUMMARY OF THE INVENTION

The present invention addresses this need by providing a novel neuro-growth factor like polypeptide called Zneu1 and related compositions and methods. Within one aspect, the present invention provides an isolated polynucleotide encoding a mammalian polypeptide termed Zneu1. The mature human Zneu1 polypeptide is comprised of a sequence of amino acids approximately 254 amino acids long. Amino acid residue 20 of SEQ ID NO: 2, a threonine, is the initial amino acid of the mature polypeptide. Thus, it is believed that amino residues 1-19 comprise a signal sequence, and the mature Zneu1 polypeptide is represented by the amino acid sequence comprised of residues 20-254. The mature Zneu1 polypeptide is further represented by SEQ ID NO: 3. Mouse Zneu1 is defined by SEQ ID NOs:18 and 19. Having a signal sequence of amino acid residues 1-23, and the mature mouse Zneu1 is from 24-278 represented by SEQ ID NO: 24. Within an additional embodiment, the polypeptide further comprises an affinity tag. Within a further embodiment, the polynucleotide is DNA.

Within a second aspect of the invention there is provided an expression vector comprising (a) a transcription promoter; (b) a DNA segment encoding Zneu1 polypeptide, and (c) a transcription terminator, wherein the promoter, DNA segment, and terminator are operably linked.

Within a third aspect of the invention there is provided a cultured eukaryotic cell into which has been introduced an expression vector as disclosed above, wherein said cell expresses a protein polypeptide encoded by the DNA segment.

Within a further aspect of the invention there is provided a chimeric polypeptide consisting essentially of a first portion and a second portion joined by a peptide bond. The first portion of the chimeric polypeptide consists essentially of (a) a Zneu1 polypeptide as shown in SEQ ID NO: 2 (b) allelic variants of SEQ ID NO:2; and (c) protein polypeptides that are at least 90% identical to (a) or (b). The second portion of the chimeric polypeptide consists essentially of another polypeptide such as an affinity tag. Within one embodiment the affinity tag is an immunoglobulin F_{c} polypeptide. The invention also provides expression vectors encoding the chimeric polypeptides and host cells transfected to produce the chimeric polypeptides.

Within an additional aspect of the invention there is provided an antibody that specifically binds to a Zneu1 polypeptide as disclosed above, and also an anti-idiotypic antibody which neutralizes the antibody to a Zneu1 polypeptide.

In addition to the above, the present invention is also directed domains of the polypeptide including SEQ ID NOs:8, 9, 10, 11, 12, 13, 14, 15, and 16.

An additional embodiment of the present invention relates to a peptide or polypeptide which has the amino acid sequence of an epitope-bearing portion of a Zneu1 polypeptide having an amino acid sequence described above. Peptides or polypeptides having the amino acid sequence of an epitope-bearing portion of a Zneu1 polypeptide of the present invention include portions of such polypeptides with at least nine, preferably at least 15 and more preferably at least 30 to 50 amino acids, although epitope-bearing polypeptides of any length up to and including the entire amino acid sequence of a polypeptide of the present invention described above are also included in the present invention. Specific examples of said polypeptides are defined by the amino acid sequences of SEQ ID NOs:20-23. Also claimed are any of these polypeptides that are fused to another polypeptide or carrier molecule.

Another embodiment of the present invention relates to method for producing an antibody which binds to a peptide or polypeptide defined by SEQ ID NOs: 2-3,8, 9, 11-16, and 19-24 or to a peptide or polypeptide which is at least 90% identical to said peptide or polypeptide comprising inoculating an animal with said peptide or polypeptide or with a nucleic acid which encodes said peptide or polypeptide, wherein said animal produces antibodies to said peptide or polypeptide; and isolating said antibody.

These and other aspects of the invention will become evident upon reference to the following detailed description.

### DETAILED DESCRIPTION OF THE INVENTION

The teachings of all of the references cited herein are incorporated in their entirety by reference.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems.

“Operably linked”, when referring to DNA segments, indicates that the segments are arranged so that they function in concert for their intended purposes, e.g. transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “soluble protein” is a protein polypeptide that is not bound to a cell membrane.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is about 0.02 M or less at pH 7 and the temperature is at least about 60° C. As previously noted, the isolated polynucleotides of the present invention include DNA and RNA. Methods for isolating DNA and RNA are well known in the art. Total RNA can be prepared using guanidine HCl extraction followed by isolation by centrifugation in a CsCl gradient, Chirgwin et al., Biochemistry 18:52-94 (1979). Poly (A)⁺ RNA is prepared from total RNA using the method of Aviv and Leder, Proc. Natl. Acad. Sci. USA 69:1408-1412 (1972). Complementary DNA (cDNA) is prepared from poly(A)⁺ RNA using known methods. Polynucleotides encoding Zneu1 polypeptides are then identified and isolated by, for example, hybridization or PCR.

The polynucleotides of the present invention can be synthesized using DNA synthesizer. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick, Bernard R. and Jack J. Pasternak, Molecular Biotechnology, Principles & Applications of Recombinant DNA, (ASM Press, Washington, D.C. 1994), Itakura, K. et al. Synthesis and use of synthetic oligonucleotides. Annu. Rev. Biochem. 53 : 323-356 (1984), and Climie, S. et al. Chemical synthesis of the thymidylate synthase gene. Proc. Natl. Acad. Sci. USA 87 :633-637 (1990).

Those skilled in the art will recognize that the sequences disclosed in SEQ ID NOS:1, 2 and 3 represent a single allele of the human. Allelic variants of these sequences can be cloned by probing cDNA or genomic libraries from different individuals according to standard procedures.

The present invention further provides counterpart proteins and polynucleotides from other species (“species orthologs”). Of particular interest are Zneu1 polypeptides from other mammalian species, including murine, porcine, ovine, bovine, canine, feline, equine, and other primates. Species orthologs of the human Zneu1 protein can be cloned using information and compositions provided by the present invention in combination with conventional cloning techniques. For example, a cDNA can be cloned using mRNA obtained from a tissue or cell type that expresses the protein. Suitable sources of mRNA can be identified by probing Northern blots with probes designed from the sequences disclosed herein. A library is then prepared from mRNA of a positive tissue or cell line. A protein-encoding cDNA can then be isolated by a variety of methods, such as by probing with a complete or partial human or mouse cDNA or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to the protein. Similar techniques can also be applied to the isolation of genomic clones. As used and claimed the language “an isolated polynucleotide which encodes a polypeptide, said polynucleotide being defined by SEQ ID NO: 2” includes all allelic variants and species orthologs of the polypeptide of SEQ ID NO:2.

The present invention also provides isolated protein polypeptides that are substantially homologous to the polypeptide of SEQ ID NO: 3 and its species orthologs. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. The term “substantially homologous” is used herein to denote polypeptides having 50%, preferably 60%, more preferably at least 80%, sequence identity to the sequence shown in SEQ ID NO:2,or its species orthologs. Such polypeptides will more preferably be at least 90% identical, and most preferably 95% or more identical to SEQ ID NO:3,or its species orthologs. Percent sequence identity is determined by conventional methods. See, for example, Altschul et al., Bull. Math. Bio. 48: 603-616 (1986) and Henikoff and Henikoff, Proc. Natl. Acad. Sci. USA 89:10915-10919 (1992). Briefly, two amino acid sequences are aligned to optimize the alignment scores using a gap opening penalty of 10, a gap extension penalty of 1, and the “blossom 62” scoring matrix of Henikoff and Henikoff (ibid.) as shown in Table 1 (amino acids are indicated by the standard one-letter codes). The percent identity is then calculated as:

$$\frac{Total number of identical matches}{\begin{matrix}\begin{matrix}[ length of the longer sequence plus the \\ number of gaps introduced into the longer\end{matrix} \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Substantially homologous proteins and polypeptides are characterized as having one or more amino acid substitutions, deletions or additions. These changes are preferably of a minor nature, that is conservative amino acid substitutions (see Table 2) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A, Nilsson et al., EMBO J. 4:1075, (1985); Nilsson et al., Methods Enzymol. 198:3, (1991), glutathione S transferase, Smith and Johnson, Gene 67:31, (1988), or other antigenic epitope or binding domain. See, in general Ford et al., Protein Expression and Purification 2: 95-107, (1991). DNAs encoding affinity tags are available from commercial suppliers (e.g., Pharmacia Biotech, Piscataway, N.J.).

TABLE 2
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Essential amino acids in the polypeptides of the present invention can be identified according to procedures known in the art, such as site-directed mutagenesis or alanine-scanning mutagenesis, Cunningham and Wells, Science 244, 1081-1085, (1989); Bass et al., Proc. Natl. Acad. Sci. USA 88:4498-4502, (1991). In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand-protein interaction can also be determined by analysis of crystal structure as determined by such techniques as nuclear magnetic resonance, crystallography or photoaffinity labeling. See, for example, de Vos et al., Science 255:306-312, (1992); Smith et al., J. Mol. Biol. 224:899-904, (1992); Wlodaver et al., FEBS Lett. 309:59-64, (1992). The identities of essential amino acids can also be inferred from analysis of homologies with related proteins.

Multiple amino acid substitutions can be made and tested using known methods of mutagenesis and screening, such as those disclosed by Reidhaar-Olson and Sauer, Science 241:53-57, (1988) or Bowie and Sauer, Proc. Natl. Acad. Sci. USA 86:2152-2156, (1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display, e.g., Lowman et al., Biochem. 30:10832-10837, (1991); Ladner et al., U.S. Pat. No. 5,223,409; Huse, WIPO Publication WO 92/06204, and region-directed mutagenesis, Derbyshire et al., Gene 46:145, (1986); Ner et al., DNA 7:127, (1988)

Mutagenesis methods as disclosed above can be combined with high-throughput screening methods to detect activity of cloned, mutagenized proteins in host cells. Preferred assays in this regard include cell proliferation assays and biosensor-based ligand-binding assays, which are described below. Mutagenized DNA molecules that encode active proteins or portions thereof (e.g., ligand-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptides that are substantially homologous to SEQ ID NO:3 or allelic variants thereof and retain the properties of the wild-type protein. As expressed and claimed herein the language, “a polypeptide as defined by SEQ ID NO: 2” includes all allelic variants and species orthologs of the polypeptide.

The protein polypeptides of the present invention, including full-length proteins, protein fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., (1989), and Ausubel et al., ibid.

In general, a DNA sequence encoding a Zneu1 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

Another embodiment of the present invention provides for a peptide or polypeptide comprising an epitope-bearing portion of a polypeptide of the invention. The epitope of the this polypeptide portion is an immunogenic or antigenic epitope of a polypeptide of the invention. A region of a protein to which an antibody can bind is defined as an “antigenic epitope”. See for instance, Geysen, H. M. et al., Proc. Natl. Acad Sci. USA 81:3998-4002 (1984).

As to the selection of peptides or polypeptides bearing an antigenic epitope (i.e., that contain a region of a protein molecule to which an antibody can bind), it is well known in the art that relatively short synthetic peptides that mimic part of a protein sequence are routinely capable of eliciting an antiserum that reacts with the partially mimicked protein. See Sutcliffe, J. G. et al. Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer soluble peptides, especially those containing proline residues, usually are effective.

Antigenic epitope-bearing peptides and polypeptides of the invention are therefore useful to raise antibodies, including monoclonal antibodies, that bind specifically to a polypeptide of the invention. Antigenic epitope-bearing peptides and polypeptides of the present invention contain a sequence of at least nine, preferably between 15 to about 30 amino acids contained within the amino acid sequence of a polypeptide of the invention. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that react with the protein. Preferably, the amino acid sequence of the epitope-bearing peptide is selected to provide substantial solubility in aqueous solvents (i.e., the sequence includes relatively hydrophilic residues and hydrophobic residues are preferably avoided); and sequences containing proline residues are particularly preferred. All of the polypeptides shown in the sequence listing contain antigenic epitopes to be used according to the present invention, however, specifically designed antigenic epitopes include the peptides defined by SEQ ID NOs:20-24.

Polynucleotides, generally a cDNA sequence, of the present invention encode the above-described polypeptides. A cDNA sequence which encodes a polypeptide of the present invention is comprised of a series of codons, each amino acid residue of the polypeptide being encoded by a codon and each codon being comprised of three nucleotides. The amino acid residues are encoded by their respective codons as follows.

Alanine (Ala) is encoded by GCA, GCC, GCG or GCT;

Cysteine (Cys) is encoded by TGC or TGT;

Aspartic acid (Asp) is encoded by GAC or GAT;

Glutamic acid (Glu) is encoded by GAA or GAG;

Phenylalanine (Phe) is encoded by TTC or TTT;

Glycine (Gly) is encoded by GGA, GGC, GGG or GGT;

Histidine (His) is encoded by CAC or CAT;

Isoleucine (Ile) is encoded by ATA, ATC or ATT;

Lysine (Lys) is encoded by AAA, or AAG;

Leucine (Leu) is encoded by TTA, TTG, CTA, CTC, CTG or CTT;

Methionine (Met) is encoded by ATG;

Asparagine (Asn) is encoded by AAC or AAT;

Proline (Pro) is encoded by CCA, CCC, CCG or CCT;

Glutamine (Gln) is encoded by CAA or CAG;

Arginine (Arg) is encoded by AGA, AGG, CGA, CGC, CGG or CGT;

Serine (Ser) is encoded by AGC, AGT, TCA, TCC, TCG or TCT;

Threonine (Thr) is encoded by ACA, ACC, ACG or ACT;

Valine (Val) is encoded by GTA, GTC, GTG or GTT;

Tryptophan (Trp) is encoded by TGG; and

Tyrosine (Tyr) is encoded by TAC or TAT.

It is to be recognized that according to the present invention, when a cDNA is claimed as described above, it is understood that what is claimed are both the sense strand, the anti-sense strand, and the DNA as double-stranded having both the sense and anti-sense strand annealed together by their respective hydrogen bonds. Also claimed is the messenger RNA (mRNA) which encodes the polypeptides of the present invention, and which mRNA is encoded by the above-described cDNA. A messenger RNA (mRNA) will encode a polypeptide using the same codons as those defined above, with the exception that each thymine(T) is replaced by a uracil nucleotide (U).

To direct a Zneu1 polypeptide into the secretory pathway of a host cell, a secretory signal sequence (also known as a leader sequence, prepro sequence or pre sequence) is provided in the expression vector. The secretory signal sequence may be that of the protein, or may be derived from another secreted protein (e.g., t-PA) or synthesized de novo. The secretory signal sequence is joined to the Zneu1 DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are preferred hosts within the present invention. Methods for introducing exogenous DNA into mammalian host cells include calcium phosphate-mediated transfection, Wigler et al., Cell 14:725, (1978); Corsaro and Pearson, Somatic Cell Genetics 7:603, (1981): Graham and Van der Eb, Virology 52:456, (1973), electroporation, Neumann et al., EMBO J. 1:841-845, (1982), DEAE-dextran mediated transfection, Ausubel et al., eds., Current Protocols in Molecular Biology, John Wiley and Sons, Inc., NY, (1987), and liposome-mediated transfection, Hawley-Nelson et al., Focus 15:73, (1993); Ciccarone et al., Focus 15:80, (1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293, ATCC No. CRL 1573; Graham et al., J. Gen. Virol. 36:59-72, (1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978,and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. Transformation of insect cells and production of foreign polypeptides therein is disclosed by Guarino et al., U.S. Pat. No. 5,162,222; Bang et al., U.S. Pat. No. 4,775,624; and WIPO publication WO 94/06463. The use of Agrobacterium rhizogenes as a vector for expressing genes in plant cells has been reviewed by Sinkar et al., J. Biosci. (Bangalore) 11:47-58, (1987).

Fungal cells, including yeast cells, and particularly cells of the genus Saccharomyces, can also be used within the present invention, such as for producing protein fragments or polypeptide fusions. Methods for transforming yeast cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092)and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465, (1986) and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Within one aspect of the present invention, a novel protein is produced by a cultured cell, and the cell is used to screen for a receptor or receptors for the protein, including the natural receptor, as well as agonists and antagonists of the natural ligand.

PROTEIN ISOLATION:

Expressed recombinant polypeptides (or chimeric polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, (1988).

The polypeptides of the present invention can be isolated by exploitation of their properties. For example, immobilized metal ion adsorption (IMAC) chromatography can be used to purify histidine-rich proteins. Briefly, a gel is first charged with divalent metal ions to form a chelate, E. Sulkowski, Trends in Biochem. 3:1-7, (1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography, Methods in Enzymol., Vol. 182, “Guide to Protein Purification”, M. Deutscher, (ed.), Acad. Press, San Diego, (1990), pp.529-39.Alternatively, a fusion of the polypeptide of interest and an affinity tag (e.g., polyhistidine, maltose-binding protein, an immunoglobulin domain) may be constructed to facilitate purification.

Physical Structure of Zneu1

The Zneu1 polypeptide shown in SEQ ID NO: 2 has a signal peptide including amino acid residues 1-19. Amino acid residues 20-104 define a hydrophilic domain homologous to an HSMHC3W5A domain, SEQ ID NO: 17, (GenBank No. g1401159). Amino acid residues 105-135 define a domain homologous to an Epidermal Growth Factor (EGF) domain. Amino acid residues 136-177 define another domain homologous to an EGF domain; and amino acid residues 178-273 define a domain also homologous to an HSMHC3W5A domain.

However, the first EGF-like domain (EGF1) of Zneu1, SEQ ID NO: 9 which corresponds to amino acid residues 105 to 135 of SEQ ID NO: 2, is distinct from any other EGF domain in the prior art. The EGF1 in Zneu1 is about 56% similar to the HSMHC3W5A_{—}6 domain, its closest human relative.

The second EGF-like domain (EGF2) of Zneu1, SEQ ID NO: 10 which corresponds to amino acid residues 136 to 177 of SEQ ID NO: 2,is distinct from any other EGF domain in the prior art. EGF2 of Zneu1 is about 48% similar to PIR_S31101 fibrillin, its closest human relative.

The first HSMHC3W5A-like (HSM1) domain of Zneu1, SEQ ID NO: 8 which corresponds to amino acid residues 20-104 of SEQ ID NO: 2. SEQ ID NO: 8 is approximately 38% similar to HSMHC3W5A,its closest human relative.

The second HSMHC3W5A-like domain (HSM2) of Zneu1, SEQ ID NO: 11 which corresponds to amino acid residues 178-273 of SEQ ID NO: 2, is distinct from any other polypeptide in the prior art. It is about 32% similar to HSMHC3W5A_{—}6.

Uses

The tissue specificity of Zneu1 expression suggests that Zneu1 may be a growth, maintenance, or differentiation factor in the spinal cord, heart, spleen, testis, thyroid and lymph nodes.

The present invention also provides reagents which will find use in diagnostic applications. For example, the Zneu1 gene has been mapped on chromosome 9q34.3. A Zneu1 nucleic acid probe could be used to check for abnormalities in chromosome 9. In a normal chromosome 9, one would predict that a Zneu1 nucleic acid probe would hybridize to chromosome 9. If the probe does not hybridize to chromosome 9, this would indicate an abnormality in chromosome 9.

Zneu1's closest human homolog is HSMHC3W5A a gene in the HLA class III region, which is contained in a cosmid which contains Notch 4. Zneu1 is also homologous to Notch 4 in its EGF-like domains. Zneu1 may be involved in EGF-receptor pathways.

Notch Structure/Function

The original member of this gene family was the Drosophila gene Notch which controls cell fate decisions in the development of the peripheral nervous system. Notch is a cell surface receptor with a single transmembrane domain. Homologues have now been found in C. elegans (lin12 and g1p1), Xenopus, mouse and human. All members of the Notch family have large numbers of EGF-like motifs (29-39 in mouse, 10-13 in C. elegans) and three or more copies of LNR (lin12/Notch repeats) in the extracellular domain. Notch family members also contain six copies of the cdc10/SWI6 motif (also called ankyrin repeats) and a PEST protein degradation sequence in the intracellular domain. Specific EGF repeats (Drosophila repeats 11 and 12) are involved in ligand binding. LNR may be regulatory domains which bind ligand when high ligand concentrations exist and cause decreased activity of Notch. Cdc10/SWI6 domains are involved in protein-protein interactions with components of the Notch-activated signal transduction pathway.

Notch Bioloqy

Two different translocations led to formation of altered Notch genes resulting in an oncogenic state. The TAN-1 oncogene is a fusion of part of the β T cell receptor with a small region of the human Notch 1 extracellular domain and the entire intracellular domain. TAN-1 is an activated form of Notch which causes T-lymphoblastic leukemias. The int-3 oncogene is caused by integration of the mouse mammary tumor virus into the Notch 4 gene resulting in expression of the intact intracellular domain. Int-3 also is an activated form of Notch which leads to mammary carcinoma.

The function of Notch family members has been extensively studied in Drosophila and C. elegans. These proteins control binary decisions that depend on cell-cell interactions. Notch proteins act consistent with their proposed role as a receptor. Gain-of-function and loss-of-function Notch alleles result in opposite cell fate decisions. Notch receptors and their ligands play important roles in lateral inhibition, the process whereby signaling between neighboring cells is amplified by a feedback loop between Notch and its ligand. This process results in increased receptor activity in some cells and increased ligand activity in others leading to the distinction between signaling cells and receiving cells.

It has recently been shown that the expression of an activated form of Notch1 in developing T cells of the mouse leads to both an increase in CD8 lineage T cells and a decrease in CD4 lineage T cells. Expression of activated Notch permits the development of mature CD8 lineage thymocytes even in the absence of class I major histocompatability complex (MHC) proteins, ligands that are normally required for the development of these cells. However, activated Notch is not sufficient to promote CD8 when both class I and class II MHC are absent. These results implicate Notch as a participant in the CD4 versus CD8 lineage decision. Robey, E. et al. Cell 87: 483-492 (1996).

Mutations in a gene region called CADASIL (for cerebral autosomal dominant arteriopathy with subcortical infarcts and leukoencephalopathy) on chromosome 19 are associated with a type of stroke and dementia whose key features include recurrent subcortical ischaemic events and vascular dementia. Notch3 has been mapped to this region, and mutations in CADASIL patients indicate that Notch3 could be the defective protein in CADASIL patients, Joutel, A. et al. Nature 383:707-710 (1996).

Notch Ligands

There is also a conserved family of ligands for the Notch receptor family. Multiple ligands are able to activate the same receptor. For example, delta and serrate each act as ligands for Drosophila Notch. These ligands all contain EGF repeats (from 1-14), a DSL domain (delta, serrate, lag-2) and a transmembrane domain. Therefore, receptor and ligand are homologous to one another. In addition, receptor and ligand are often coexpressed and are associated with each other in vesicles.

Zneu1 Structure

Zneu1 is similar to Notch and its ligands in having two EGF repeats. However, it has a small number of EGF repeats and lacks a membrane spanning domain, lin12/Notch domains and ankyrin repeats. Based on structure/function experiments of Notch, one would predict that Zneu1 would antagonize Notch function. If the EGF repeats in zneul could bind receptor, it could inhibit ligand binding on neighboring cells. Furthermore, Zneu1 may have its own target receptor for which it would be an agonist.

Zneu1 Tissue Distribution/Multiple mRNA Sizes

Zneu1 is widely expressed in adult human tissues. Zneu1 is most highly expressed in heart, placenta, spleen, testis, thyroid, spinal cord and lymph node. Dot blots indicate that Zneu1 is also expressed in a variety of fetal tissues. There are at least three mRNA sizes:

1.3 kb mRNA only in brain and testis 1.7 kb only in lymph node

1.3+1.7 in multiple tissues

2.4 kb only in placenta

Since the sequence of Zneu1 is from the 1.3 kb mRNA in brain, it is difficult to predict what types of molecules the larger transcripts encode. It is possible that larger forms could encode soluble Zneu1 proteins with more EGF repeats and other domains observed in Notch or Notch ligands. Alternatively, the extra sequences could encode transmembrane and intracellular domains.

Possible Relationship to Notch Function

It is difficult to predict whether Zneu1 will act as a Notch ligand or to antagonize the activity of other Notch ligands by competing for receptor binding. Zneu1 may alter the binary decisions in differentiation of stem cells into specific lineages or may alter the cell fate decisions of adjacent cells.

Alternatively, Zneu1 may have nothing to do with Notch. Many proteins have EGF repeats. Zneu1 may act as a growth factor for a different class of receptor.

Other Possible Roles

role in breast cancer (EGF-receptor is overexpressed in many breast cancers)

role in glioblastomas, pituitary adenomas.

Mapping Data

Zneu1 maps to human chromosome 9q34.3, in the same chromosomal band as Notch1. It is of interest that Notch4 and HSMHC3W5A are also linked at the MHC III locus, i.e., duplication of an authentic Notch receptor and a 2 EGF-repeat novel protein.

Therapeutic utility

Zneu1 and its antagonists can be used as therapeutic reagents for the following.

1. Alzheimer's disease

The Sell2 gene was identified as a suppresser of a lin12 gain-of-function mutant. Sell2 is a homolog of a positional cloned human early-onset familial Alzheimer's disease gene. Therefore, Zneu1 could affect a pathway affecting this disease and it is expressed in brain, albeit at lower levels than most other tissues.

2. Cancer

There are a number of chromosomal rearrangements associated with breakpoints at 9q34 including Non-hodgkin's lymphoma and acute myeloid leukemia. A probe for Zneu1 which does not properly hybridize to chromosome 9q34 would indicate an abnormality of chromosome 9 and would indicate a possible predilection of the individual for developing cancer.

Given the possible association with Notch 4, an endothelial-specific gene, Zneu1 could be involved in promoting or inhibiting endothelial cell tumors such as hemangiopericytomas? Another possibility is in angiogenesis since blocking a tumor's blood supply would be an effective cancer treatment.

Given the tissues where Zneu1 is highly expressed, the most prevalent forms of cancer would be in the testis and lymph nodes.

3. Hematopoiesis

Moore et al (PNAS 94:4011-4016, 1997) implicated delta-like (a mammalian Notch ligand) in promoting both high-proliferative potential progenitors and in stem cell repopulation. Since Zneu1 is highly expressed in lymph node and spleen, it could either be involved in inhibiting differentiation to promote stem cell self-renewal or in determination of progenitor populations. Possible use in repopulating blood cells after chemotherapy treatment or in vitro expansion of stem cells.

4. Heart

Stimulation of myofibroblast proliferation or migration in the repair process after myocardial infarction. Recently, a frizzled homolog has been implicated in this process. There is evidence for interactions between the frizzled and Notch pathways in Drosophila.

5. Placenta

Stimulation or inhibition of various growth factor made in placenta.

6. Testis

Role in fertility or contraception

7. Spinal cord

Zneu1 may play a role in Nerve regeneration since Notch plays a role in neurogenesis in both flies and mammalian cells.

The present invention also provides reagents with significant therapeutic value. The Zneu1 polypeptide (naturally occurring or recombinant), fragments thereof, antibodies and anti-idiotypic antibodies thereto, along with compounds identified as having binding affinity to the Zneu1 polypeptide, should be useful in the treatment of conditions associated with abnormal physiology or development, including abnormal proliferation, e.g., cancerous conditions, or degenerative conditions. For example, a disease or disorder associated with abnormal expression or abnormal signaling by a Zneu1 polypeptide should be a likely target for an agonist or antagonist of the Zneu1 polypeptide.

Antibodies to the Zneu1 polypeptide can be purified and then administered to a patient. These reagents can be combined for therapeutic use with additional active or inert ingredients, e.g., in pharmaceutically acceptable carriers or diluents along with physiologically innocuous stabilizers and excipients. These combinations can be sterile filtered and placed into dosage forms as by lyophilization in dosage vials or storage in stabilized aqueous preparations. This invention also contemplates use of antibodies, binding fragments thereof or single-chain antibodies of the antibodies including forms which are not complement binding.

The quantities of reagents necessary for effective therapy will depend upon many different factors, including means of administration, target site, physiological state of the patient, and other medications administered. Thus, treatment dosages should be titrated to optimize safety and efficacy. Typically, dosages used in vitro may provide useful guidance in the amounts useful for in vivo administration of these reagents. Animal testing of effective doses for treatment of particular disorders will provide further predictive indication of human dosage. Methods for administration include oral, intravenous, peritoneal, intramuscular, or transdermal administration. Pharmaceutically acceptable carriers will include water, saline, buffers to name just a few. Dosage ranges would ordinarily be expected from 1 μg to 1000 μg per kilogram of body weight per day. However, the doses by be higher or lower as can be determined by a medical doctor with ordinary skill in the art. For a complete discussion of drug formulations and dosage ranges see Remington's Pharmaceutical Sciences, 17^{th} Ed., (Mack Publishing Co., Easton, Pa., 1990), and Goodman and Gilman's: The Pharmacological Bases of Therapeutics, 9^{th} Ed. (Pergamon Press 1996).

Nucleic Acid-Based Therapeutic Treatment

If a mammal has a mutated or lacks a Zneu1 gene, the Zneu1 gene can be introduced into the cells of the mammal. In one embodiment, a gene encoding a Zneu1 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector [Kaplitt et al., Molec. Cell. Neurosci.,2:320-330 (1991)], an attenuated adenovirus vector, such as the vector described by Stratford-Perricaudet et al., J. Clin. Invest., 90 :626-630 (1992), and a defective adeno-associated virus vector [Samulski et al., J. Virol., 61:3096-3101 (1987); Samulski et al. J. Virol., 63:3822-3828 (1989)].

In another embodiment, the gene can be introduced in a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995 by Dougherty et al.; and Blood, 82:845 (1993).

Alternatively, the vector can be introduced by lipofection in vivo using liposomes. Synthetic cationic lipids can be used to prepare liposomes for in vivo transfection of a gene encoding a marker [Felgner et al., Proc. Natl. Acad. Sci. USA, 84:7413-7417 (1987); see Mackey et al., Proc. Natl. Acad. Sci. USA, 85:8027-8031 (1988)]. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter [see, e.g., Wu et al., J. Biol. Chem., 267:963-967 (1992); Wu et al., J. Biol. Chem., 263:14621-14624 (1988)].

ANTIBODIES

Zneu1 polypeptides can also be used to prepare antibodies that specifically bind to Zneu1 epitopes, peptides or polypeptides. The Zneu1 polypeptide or a fragment thereof serves as an antigen (immunogen) to inoculate an animal and elicit an immune response. Suitable antigens would be the Zneu1 polypeptide encoded by SEQ ID NO:2 or 3 or at least a contiguous 9 amino acid fragment thereof. Antibodies generated from this immune response can be isolated and purified as described herein. Methods for preparing and isolating polyclonal and monoclonal antibodies are well known in the art. See, for example, Current Protocols in Immunology, Cooligan, et al. (eds.), National Institutes of Health, (John Wiley and Sons, Inc., 1995); Sambrook et al., Molecular Cloning: A Laboratory Manual, Second Edition (Cold Spring Harbor, N.Y., 1989); and Hurrell, J. G. R., Ed., Monoclonal Hybridoma Antibodies: Techniques and Applications (CRC Press, Inc., Boca Raton, Fla., 1982).

As would be evident to one of ordinary skill in the art, polyclonal antibodies can be generated from inoculating a variety of warm-blooded animals such as horses, cows, goats, sheep, dogs, chickens, rabbits, mice, and rats with a Zneu1 polypeptide or a fragment thereof. The immunogenicity of a Zneu1 polypeptide may be increased through the use of an adjuvant, such as alum (aluminum hydroxide) or Freund's complete or incomplete adjuvant. Polypeptides useful for immunization also include fusion polypeptides, such as fusions of Zneu1 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to Zneu1 protein or peptide, and selection of antibody display libraries in phage or similar vectors (for instance, through use of immobilized or labeled Zneu1 protein or peptide). Genes encoding polypeptides having potential Zneu1 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the Zneu1 sequences disclosed herein to identify proteins which bind to Zneu1. These “binding proteins” which interact with Zneu1 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as Zneu1 “antagonists” to block Zneu1 binding and signal transduction in vitro and in vivo. These anti-Zneu1 binding proteins would be useful for down regulating the effect of Zneu1.

Antibodies are determined to be specifically binding if: 1) they exhibit a threshold level of binding activity, and/or 2) they do not significantly cross-react with related polypeptide molecules. First, antibodies herein specifically bind if they bind to a Zneu1 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis.

Second, antibodies are determined to specifically bind if they do not significantly cross-react with related polypeptides. Antibodies do not significantly cross-react with related polypeptide molecules, for example, if they detect Zneu1 but not known related polypeptides using a standard Western blot analysis (Ausubel et al., ibid.). Examples of known related polypeptides are orthologs, proteins from the same species that are members of a protein family (e.g. IL-16), Zneu1 polypeptides, and non-human Zneu1. Moreover, antibodies may be “screened against” known related polypeptides to isolate a population that specifically binds to the inventive polypeptides. For example, antibodies raised to Zneu1 are adsorbed to related polypeptides adhered to insoluble matrix; antibodies specific to Zneu1 will flow through the matrix under the proper buffer conditions. Such screening allows isolation of polyclonal and monoclonal antibodies non-crossreactive to closely related polypeptides, Antibodies: A Laboratory Manual, Harlow and Lane (eds.) (Cold Spring Harbor Laboratory Press, 1988); Current Protocols in Immunology, Cooligan, et al. (eds.), National Institutes of Health (John Wiley and Sons, Inc., 1995). Screening and isolation of specific antibodies is well known in the art. See, Fundamental Immunology, Paul (eds.) (Raven Press, 1993); Getzoff et al., Adv. in Immunol. 43: 1-98 (1988); Monoclonal Antibodies: Principles and Practice, Goding, J.W. (eds.), (Academic Press Ltd., 1996); Benjamin et al., Ann. Rev. Immunol. 2: 67-101 (1984).

A variety of assays known to those skilled in the art can be utilized to detect antibodies which specifically bind to Zneu1 proteins or peptides. Exemplary assays are described in detail in Antibodies: A Laboratory Manual, Harlow and Lane (Eds.) (Cold Spring Harbor Laboratory Press, 1988). Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant Zneu1 protein or polypeptide.

Antibodies to Zneu1 may be used for tagging cells that express Zneu1; for isolating Zneu1 by affinity purification; for diagnostic assays for determining circulating levels of Zneu1 polypeptides; for detecting or quantitating soluble Zneu1 as marker of underlying pathology or disease; in analytical methods employing FACS; for screening expression libraries; for generating anti-idiotypic antibodies; and as neutralizing antibodies or as antagonists to block Zneu1 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to Zneu1 or fragments thereof may be used in vitro to detect denatured Zneu1 or fragments thereof in assays, for example, Western Blots or other assays known in the art.

An additional embodiment of the present invention relates to a peptide or polypeptide which has the amino acid sequence of an epitope-bearing portion of a Zneu1 polypeptide having an amino acid sequence described above. Peptides or polypeptides having the amino acid sequence of an epitope-bearing portion of a Zneu1 polypeptide of the present invention include portions of such polypeptides with at least nine, preferably at least 15 and more preferably at least 30 to 50 amino acids, although epitope-bearing polypeptides of any length up to and including the entire amino acid sequence of a polypeptide of the present invention described above are also included in the present invention. Examples of said polypeptides are defined by the amino acid sequences of SEQ ID NOs:20-23. Also claimed are any of these polypeptides that are fused to another polypeptide or carrier molecule.

The invention is further illustrated by the following non-limiting examples.

### EXAMPLE 1

### Cloning of Zneu1

Zneu1 was identified from expressed sequence tag (EST) SEQ ID NO: 4. The cDNA clone containing the EST was discovered in a brain cDNA library which contained the EST. The cDNA was isolated from E. coli transfected with the plasmid and then streaked out on an LB 100 μg/ml ampicillin and 100 μg/ml methicillin plate. The cDNA insert was sequenced. The insert was determined to be 1514 base pairs long with a 274 amino acid open reading frame and a putative 19 amino acid signal peptide.

### EXAMPLE 2

### Northern Blot Analysis

Human multiple tissue blots 1,2,3 (Clontech) were probed to determine the tissue distribution of Zneu1. A HindIII/NotI fragment containing the entire Zneu1 coding region was generated from the isolated CDNA clone and used for the probe. A plasmid prep of the clone was prepared from a 5 ml LB 100 μg/ml ampicillin overnight culture at 37° using the QIAprep Spin Miniprep Kit (Qiagen). 20 μl out of 100 μl were digested with 3 μl of NEB Buffer 3, 10 units of HindIII (Gibco BRL) and 10 units Not1 (New England Biolabs) in a 30 μl reaction at 37° C. for 2 hours. The digest was electrophoresed on a 0.8% TBE agarose gel and the fragment was cut out. The DNA was extracted from the gel slab with a QIAquick Gel Extraction Kit (Qiagen). 25 ng of this DNA was labeled with P³² using the Multiprime DNA Labeling System (Amersham) and unincorporated radioactivity was removed with a NucTrap Probe Purification Column (Stratagene). Multiple tissue northerns and a human RNA master blot were prehybridized 3 hours with 10 ml ExpressHyb Solution and added to blots. Hybridization was carried out overnight at 42° C. with a 10 ml solution of probe containing a concentration of 2×10⁶/ml of probe to which 1 mg of salmon sperm DNA was added which had been boiled for 5 minutes and then iced 1 minute and added to 10 ml of ExpressHyb Solution (Clontech). Initial wash conditions were as follows: 2×SSC, 0.05% SDS RT for 40 minutes with several changes of solution then 0.1×SSC, 0.1% SDS at 65° C. for 40 minutes, 1 solution change. Blots were than exposed to film a −80° C. There was cross hybridization/background so blots were further washed at 72° C. then 65° C. with 0.1%×SSC, 0.1% SDS for 1 hour each.

The results showed that Zneu1 is widely expressed in adult tissues. Zneu1 is highly expressed in heart, placenta, spleen, testis, thyroid, spinal cord and lymph node. There are at least three mRNA sizes:

1.3 kb mRNA only in brain and testis;

1.4 kb only in lymph node;

1.5+1.7 kb in multiple tissues; and

2.4 kb only in placenta.

### EXAMPLE 3

### Chromosomal Assignment and Placement of Zneu1.

Zneu1 was mapped to chromosome 9 using the commercially available “GeneBridge 4 Radiation Hybrid Panel” (Research Genetics, Inc., Huntsville, Ala.). The GeneBridge 4 Radiation Hybrid Panel contains PCRable DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of Zneu1 with the “GeneBridge 4 RH Panel”, 20 μl reactions were set up in a PCRable 96-well microtiter plate (Stratagene, La Jolla, Calif.) and used in a “RoboCycler Gradient 96” thermal cycler (Stratagene). Each of the 95 PCR reactions consisted of 2 μl 10× KlenTaq PCR reaction buffer (CLONTECH Laboratories, Inc., Palo Alto, Calif.), 1.6 μl dNTPs mix (2.5 mM each, PERKIN-ELMER, Foster City, Calif.), 1 μl sense primer, SEQ ID NO: 6, 1 μl antisense primer, SEQ ID NO: 7, 2 μl “RediLoad” (Research Genetics, Inc., Huntsville, Ala.), 0.4 μl 50× Advantage KlenTaq Polymerase Mix (Clontech Laboratories, Inc.), 25 ng of DNA from an individual hybrid clone or control and × μl ddH20 for a total volume of 20 μl. The reactions were overlaid with an equal amount of mineral oil and sealed. The PCR cycler conditions were as follows: an initial 1 cycle 5 minute denaturation at 95° C., 35 cycles of a 1 minute denaturation at 95° C., 1 minute annealing at 70° C. and 1.5 minute extension at 72° C., followed by a final 1 cycle extension of 7 minutes at 72° C. The reactions were separated by electrophoresis on a 2% agarose gel (Life Technologies, Gaithersburg, Md.).

The results showed that Zneu1 maps 529.80 cR_{—}3000 from the top of the human chromosome 9 linkage group on the WICGR radiation hybrid map, 7.90 cR_{—}3000 distal of framework marker D9S158. This positions Zneu1 in the 9q34.3 region on the integrated LDB chromosome 9 map (The Genetic Location Database, University of Southhampton, WWW server: http://cedar.genetics. soton.ac.uk/public_html/).

We claim:

1. An isolated polynucleotide which encodes a mammalian Zneu1 polypeptide wherein said polynucleotide encodes a polypeptide selected from the group SEQ ID NOs:2-3,8, 9, 11-16, and 19-24 or a polypeptide which is at least 90% identical to the polypeptides of said group and which retain the activity of said polypeptides.

2. An isolated polynucleotide which encodes a peptide or polypeptide having at least 15 amino acid residues comprised of an epitope-bearing portion of a polypeptide of SEQ ID NOs: 2-3,8, 9, 11-16, and 19-24 or a polypeptide which is at least 90% identical to said polypeptides.

3. The isolated polynucleotide of claim 2 wherein the peptide or polypeptide is selected from the group consisting of SEQ ID NOs: 2-3,8, 9, 11-16, and 19-24 or a polypeptide which is at least 90% identical to said polypeptides.

4. The polynucleotide of claim 2 wherein the peptide or polypeptide is fused to a carrier polypeptide or other carrier molecule.

5. An expression vector comprising the following operably linked elements:
a transcription promoter;
a DNA segment which encodes a Zneu1 polypeptide or a peptide or polypeptide which contains an epitope-bearing region of a Zneu1 polypeptide; and a transcription terminator.

6. An expression vector comprising the following operably linked elements:
(a) a transcription promoter;
(b) a DNA segment encoding a chimeric polypeptide, wherein said chimeric polypeptide consists essentially of a first portion and a second portion joined by a peptide bond, said first portion being comprised of a mammalian polypeptide, said polypeptide being the amino acid sequences of SEQ ID NOs: 2-3,8, 9, 11-16, and 19-24 and said second portion being a second polypeptide or protein.
(c) a transcription terminator.

7. An isolated Zneu1 polypeptide selected from the group of amino acid sequences consisting of SEQ ID NOs: 2-3,8, 9, 11-16, and 19-24 or a polypeptide which is at least 90% identical to said polypeptides.

8. An isolated peptide or polypeptide having at least 15 amino acid residues comprised of an epitope-bearing portion of a polypeptide of SEQ ID NOs: 2-3,8, 9, 11-16, and 19-24 or is at least 90% identical to said epitope bearing portion.

9. The isolated peptide or polypeptide of claim 8 wherein the epitope-bearing portion is selected from the group of amino acid sequence consisting of SEQ ID NOs:20-23 or a peptide or polypeptide which is at least 90% identical to said epitope bearing portion.

10. An antibody, antibody fragment or single-chain antibody that specifically binds to a mammalian polypeptide, said polypeptide being defined by the amino acid sequences of SEQ ID NOs: 2-3,8, 9, 11-16, and 19-24.

11. An antibody of claim 10 wherein said antibody is either monoclonal or polyclonal.

12. The antibody, antibody fragment or single-chain antibody of claim 10 wherein said antibody, antibody fragment or single-chain antibody is humanized.

13. A method for producing an antibody which binds to a peptide or polypeptide defined by SEQ ID NOs: 2-3,8, 9, 11-16, and 19-24 or to a peptide or polypeptide which is at least 90% identical to said peptide or polypeptide comprising inoculating an animal with said peptide or polypeptide or with a nucleic acid which encodes said peptide or polypeptide, wherein said animal produces antibodies to said peptide or polypeptide; and
isolating said antibody.

14. The antibody of claim 13 wherein said antibody is either a polyclonal or monoclonal antibody.

15. An anti-idiotypic antibody, anti-idiotypic antibody fragment or anti-idiotypic single-chain antibody which binds to an antibody, an antibody fragment or single-chain antibody of peptide or polypeptide defined by SEQ ID NOs: 2-3,8, 9, 11-16, and 19-24 or to a peptide or polypeptide which is at least 90% identical to said peptide or polypeptide
